(19) United States
(12) Patent Application Publication
(10) Pub. No.: US 2026/0283843 A1
(43) Pub. Date: Sep. 24, 2026

(54) SAFETY-ENHANCED ADJUSTABLE BABY BELLY BELT FOR STOMACH DISTRESS, COLIC AND GAS RELIEF

(71) Applicant: Wisit Chantawarang, Gatineau (CA)

(72) Inventor: Wisit Chantawarang, Gatineau (CA)

(21) Appl. No.: 19/087,971

(22) Filed: Mar. 24, 2025

(51) Int. Cl. A61F 7/02; A61F 7/00

(52) U.S. Cl. CPC A61F 7/02; A61F 2007/0022; A61F 2007/0088; A61F 2007/0096; A61F 2007/0219; A61F 2007/022; A61F 2007/023; A61F 2007/0242; A61F 2007/0285

(57) ABSTRACT

A system of an extendable baby belly belt for stomachache, colic and gas relief is provided. The system comprises a fabric belt for placement about a human midsection and a transparent soft pack held by the belt. The system also comprises a first quantity of gel beads held inside the pack that when heated retain and radiate heat. The system also comprises a second quantity of safety beads held inside the pack that when heated change color upon reaching a certain temperature and serve as a warning upon changing color to remove the belt and the pack from a source of the heat. The system is directed for use with children between the ages of two months to four years. In an embodiment, the safety beads change in color from black to red.

### CROSS REFERENCE TO RELATED APPLICATIONS

None

### FIELD OF THE DISCLOSURE

The present disclosure is in the field of childcare products. More particularly, the present disclosure provides systems and methods of adding safety beads to gel beads used to provide warmth to a child midsection experiencing distress, the beads changing color upon reaching a threshold temperature during warming and providing caution to parents.

### BACKGROUND

Infants and young children often experience stomach issues such as gas, reflux, and constipation. These problems are common and often manageable with lifestyle changes and medical advice when necessary. Swallowed air during feeding or crying can lead to gas buildup, causing fussiness, grunting, and pulling knees towards the chest. Reflux or spitting up result from stomach contents backing up into the esophagus. This is often harmless but can cause irritation and discomfort. Infrequent or hard stools can be a sign of constipation, which can be managed with dietary changes and medication in some cases. Loose, watery stools can be caused by viral or bacterial infections, food intolerances, or medications.

Infantile colic is characterized by excessive crying, often in the evenings, and can be a sign of gas or other digestive discomfort. Some babies can be sensitive to certain foods, such as dairy, leading to digestive upset. Pyloric stenosis is a condition where the pylorus (the muscle at the end of the stomach) thickens and narrows, preventing food from passing into the small intestine, leading to forceful vomiting.

Various methods and systems have previously been developed to address stomach issues in infants and young children. Parents may encourage their child to lie down and rest. They may offer clear fluids such as water or electrolyte solutions. Parents may introduce small amounts of bland foods like rice, applesauce, or crackers. They may apply a warm compress or heating pad to the stomach to soothe cramps or gently massage the child's belly to help with gas and indigestion.

Parents are advised to limit or avoid foods and drinks that are known to upset the stomach, such as carbonated beverages, citrus, dairy products, fried or greasy foods, and high-fat foods. They may teach their child deep breathing exercises to help relax their stomach muscles.

### BRIEF DESCRIPTION OF THE FIGURES

FIG. 1 is an image of a system of a safety-enhanced adjustable baby belly belt for stomach distress, colic and gas relief according to an embodiment of the present disclosure.

FIG. 2 is an image of a system of a safety-enhanced adjustable baby belly belt for stomach distress, colic and gas relief according to an embodiment of the present disclosure.

FIG. 3 is an image of a system of a safety-enhanced adjustable baby belly belt for stomach distress, colic and gas relief according to an embodiment of the present disclosure.

### DETAILED DESCRIPTION

Systems and methods described herein provide safety beads that change color with heated and upon reaching a threshold temperature. Such safety beads may be used with an adjustable baby belly belt intended to relieve stomach distress, colic and gas relief. The fabric belt is configured to hold a transparent, soft gel pack containing gel beads and the safety beads for retaining and radiating heat and safety beads for alerting the parent of excessive heat. The child's parent heats up the gel pack first before applying the pack to the child's midsection area. Most commonly the parent heats up the gel pack by submerging it in hot water or placing it into a microwave oven.

The safety beads accompanying the gel beads change color when heated beyond a predetermined safe temperature, in an embodiment 65 degrees Celsius. The belt is wrapped gently about the midsection of a child between the ages of two months to four years. It is unsafe for the gel pack to be excessively heated. The changing of the color indicating reaching of the heat threshold provides parents and child caregivers with clear, instant warning to prevent burns and keep the child safe.

The purpose of gel beads is to retain and radiate heat. The purpose of the safety beads provided herein is to be mixed with the gel beads and change color upon reaching the predetermined temperature threshold. The pack in which the gel beads and safety beads are sealed may be made of plastic or nylon and is soft and flexible. The gel pack weighs between 200-220 grams. The pack may be of dimensions about 5 inches by 7.5 inches and fits inside a pouch in the cloth baby belt. The baby belt is adjustable in size about the child's midsection and may be fastened by Velcro. The baby belt may have a minimum circumference of fifteen inches and may be made of cloth or polyester. With an optional extension panel attached, the belt may have a maximum circumference of 24 inches.

Gel beads are made of 65% water, 25% glycerin, and 10% Sodium Polyacrylate. They provide therapeutic and comfort effects when used as hot or cold compresses. The safety beads are made out of thermochromic pigment and general purpose polystyrene. The safety beads' purpose is to provide visual signal by changing color from its default black color to bright red when heated. The safety beads' purpose is to provide visual cue to parents not to apply an overheated gel pack to their children.

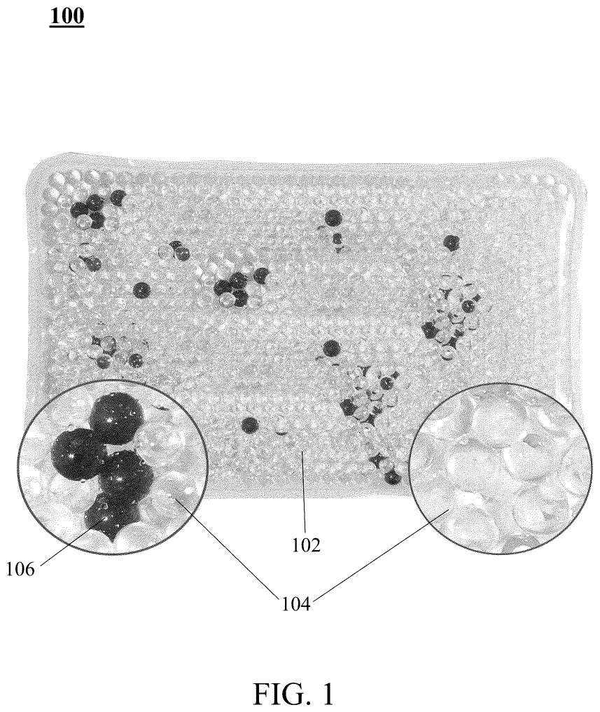

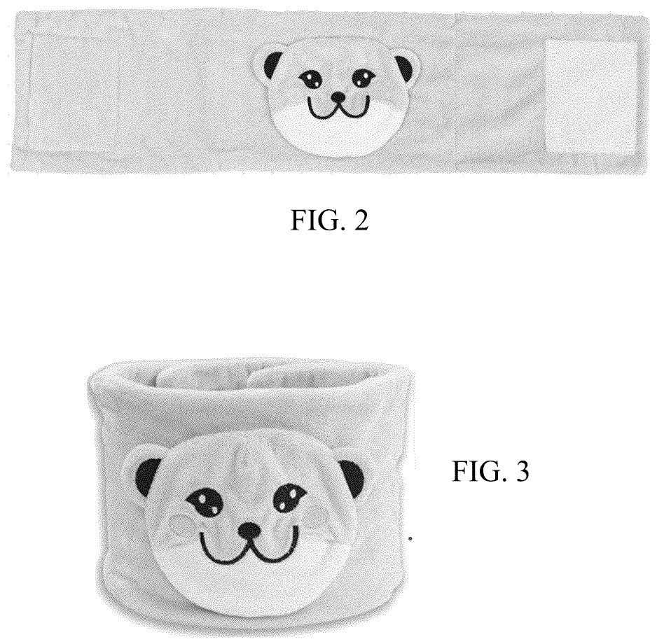

Turning to the figures, FIG. 1 is an image of a gel pack for use in a safety-enhanced adjustable baby belly belt according to an embodiment of the present disclosure. A system 100 is provided including gel pack 102, gel beads 104, and safety beads 106. Gel beads 104 and safety beads 106 may be sealed inside the gel pack 102 in different quantities. The gel beads are transparent and will not change regardless of the temperature exposure. The safety beads 106 in embodiments start out black in color and when they reach the threshold temperature, in embodiments 65 degrees Celsius, they turn red and serve as a warning to parents. FIG. 1 is provided for discussion and illustration purposes only. FIG. 2 shows the baby belt in an extended view. As noted, an additional panel can be acquired. FIG. 3 shows the baby belt as it would be closed about the baby's midsection.

In embodiments, the threshold temperature for safety beads 106 changing temperature is not 65 degrees Celsius and is instead a different temperature. In embodiments, the initial color of the safety beads 106 is not black and is instead another color. In embodiments, the color of the safety beads 106 after they reach the threshold temperature is not red and is instead another color. In embodiments, the safety beads 106 and their characteristic of changing color upon reaching a temperature threshold are not used in conjunction with gel beads 104, are not used in conjunction with a gel pack 102, and are not used in conjunction with a baby belly belt as described above and may instead be used in an entirely different and unrelated application without changing the basic novelty or character of the invention or inventor's intent described and provided herein.

In an embodiment, a system of an extendable baby belly belt for stomach ache, colic and gas relief is provided. The system comprises a fabric belt for placement about a human midsection and a transparent soft pack held by the belt. The system also comprises a first quantity of gel beads held inside the pack that when heated retain and radiate heat. The system also comprises a second quantity of safety beads held inside the pack that when heated change color upon reaching a certain temperature and serve as a warning upon changing color to remove the belt and the pack from a source of the heat.

The system is directed for use with children between the ages of two months to four years. The safety beads change in color from black to red.

The gel beads are made of 65% water, 25% glycerin, and 10% Sodium Polyacrylate. The safety beads are made of thermochromic pigment and general purpose polystyrene.

The source of the heat is a microwave device. The certain temperature is 65 degrees Celsius. The system additionally provides therapeutic and comfort effects when used as a cold compress.

In another embodiment, a method of providing relief for stomach distress, colic and gas relief in infants and young children. The method comprises a first quantity of safety beads receiving placement in a container with a second quantity of gel beads. The method also comprises the safety beads receiving heating with the gel beads while inside the container. The method also comprises the safety beads visibly changing color as temperature reaches a threshold. The method also comprises the safety beads and the gel beads while inside the container receiving removal from a source of the heating based on the visible change of color.

The change of color indicates that a temperature of the safety beads has reached a safety threshold. At ambient temperature the safety beads are black and change color to red at the threshold.

The container is a sealable transparent soft pack. The method also comprises the container and both quantities, after the heating, receiving placement within a pouch in a fabric belt. The fabric belt is thereafter placed about a human midsection.

In yet another embodiment, a system for providing stomach discomfort relief for infants and small children is provided. The system comprises a first quantity of gel beads, a second quantity of safety beads, and a sealable transparent soft pack that receives insertion of the gel beads and the safety beads. The soft pack also receives placement and heating in a heating device and receives removal from the heating device based on the safety beads changing color.

The changing of color of the safety beads is an indication that a threshold temperature has been reached. The threshold temperature is about 65 degrees Celsius. After removal from the heating device, the pack is placed in a pouch area of a fabric belt. The belt with soft pack and beads is directed to placement about a human midsection area. The heating device may be a microwave oven.

What is claimed is:

1. A system of an extendable baby belly belt for stomach ache, colic and gas relief, comprising:
a fabric belt for placement about a human midsection;
a transparent soft pack held by the belt;
a first quantity of gel beads held inside the pack that when heated retain and radiate heat; and
a second quantity of safety beads held inside the pack that:
when heated change color upon reaching a certain temperature, and
serve as a warning upon changing color to remove the belt and the pack from a source of the heat.

2. The system of claim 1, wherein the system is directed for use with children between the ages of two months to four years.

3. The system of claim 1, wherein the safety beads change in color from black to red.

4. The system of claim 1, wherein the gel beads are made of 65% water, 25% glycerin, and 10% Sodium Polyacrylate.

5. The system of claim 1, wherein the safety beads are made of thermochromic pigment and general purpose polystyrene.

6. The system of claim 1, wherein the source of the heat is a microwave device.

7. The system of claim 1, wherein the certain temperature is 65 degrees Celsius.

8. The system of claim 1, wherein the system additionally provides therapeutic and comfort effects when used as a cold compress.

9. A method of providing relief for stomach distress, colic and gas relief in infants and young children, comprising:
a first quantity of safety beads receiving placement in a container with a second quantity of gel beads;
the safety beads receiving heating with the gel beads while inside the container;
the safety beads visibly changing color as temperature reaches a threshold; and
the safety beads and the gel beads while inside the container receiving removal from a source of the heating based on the visible change of color.

10. The method of claim 9, wherein the change of color indicates that a temperature of the safety beads has reached a safety threshold.

11. The method of claim 10, wherein at ambient temperature the safety beads are black and change color to red at the threshold.

12. The method of claim 9, wherein the container is a sealable transparent soft pack.

13. The method of claim 9, further comprising the container and both quantities, after the heating, receiving placement within a pouch in a fabric belt.

14. The method of claim 13, wherein the fabric belt is thereafter placed about a human midsection.

15. A system for providing stomach discomfort relief for infants and small children, comprising:
a first quantity of gel beads;
a second quantity of safety beads;
a sealable transparent soft pack that:
receives insertion of the gel beads and the safety beads;
receives placement in a heating device,
receives heating in the heating device,
receives removal from the heating device based on the safety beads changing color.

16. The system of claim 15, wherein the changing of color of the safety beads is an indication that a threshold temperature has been reached.

17. The system of claim 16, wherein the threshold temperature is about 65 degrees Celsius.

18. The system of claim 15, wherein after removal from the heating device, the pack is placed in a pouch area of a fabric belt.

19. The system of claim 18, wherein the belt with soft pack and beads is directed to placement about a human midsection area.

20. The system of claim 15, wherein the heating device is a microwave oven.
